(19) United States
(12) Patent Application Publication
(10) Pub. No.: US 2025/0160882 A1
(43) Pub. Date: May 22, 2025

(54) CANNULA FIXING DEVICE FOR INSERTION INTO BODY

(71) Applicant: THE CATHOLIC UNIVERSITY OF KOREA INDUSTRY-ACADEMIC COOPERATION FOUNDATION, Seoul (KR)

(72) Inventor: Dong Hyun JEE, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/953,354

(22) Filed: Nov. 20, 2024

(30) Foreign Application Priority Data
Nov. 22, 2023 (KR) 10-2023-0163577

(51) Int. Cl. A61B 17/34

(52) U.S. Cl. CPC A61B 17/34; A61B 2017/3486

(57) ABSTRACT

Disclosed is a cannula fixing device for insertion into a body including a needle having a specific length, and inserted into the body, a connection part connecting a first tube that supplies a solution to the needle, and the needle, and an expansion part that is selectively expanded from an outer peripheral surface of the needle inserted into the body.

### CROSS-REFERENCE TO RELATED APPLICATIONS

A claim for priority under 35 U.S.C. § 119 is made to Korean Patent Application No. 10-2023-0163577 filed on Nov. 22, 2023 in the Korean Intellectual Property Office, the entire contents of which are hereby incorporated by reference.

### BACKGROUND

Embodiments of the present disclosure described herein relate to a cannula fixing device for insertion into a body that may prevent complications or side effects that occur when a cannula that is inserted to maintain an intraocular pressure during a vitreous body resection procedure pierces an inner surface of a retina or is separated from the eyeball.

In general, vitrectomy is a method of removing the vitreous body by using a device that simultaneously removes contents of a posterior chamber and replaces it with a saline solution, and is performed for diabetic retina diseases accompanied by vitreous hemorrhage.

This vitrectomy is performed when bleeding occurs in the vitreous body or the retina is separated to eliminate the bleeding and reattach the separated retina.

When vitrectomy is performed, an infusion port is inserted into the eyeball to maintain the pressure in the eyeball and a surgery is performed. In this case, the infusion port has to continuously supply a saline solution, such as a physiological solution that is specific to the eyeball, to maintain the pressure in the eyeball. In particular, the role of the infusion port may be even more important because it continuously suctions the resected vitreous body.

However, the infusion port may fall out of the eyeball during the vitrectomy procedure, and because the length of the needle inserted into the eyeball is only about 4 mm, and there is no structure to prevent it from falling out on the eyeball, the needle is not fixed on the surface of the eyeball and may easily fall out when touched during a surgery. In this case, when the infusion port is separated, a pressure in an interior of the eyeball is released, causing shrinkage, and the optic nerves and optic cells are damaged as it collapses in a short period of time, and thus, there is an urgent need to develop a technology for fixing the infusion port to the eyeball.

### SUMMARY

Embodiments of the present disclosure provide a cannula fixing device for insertion into a body that prevents a tip end of a needle from contacting an inner wall thereof when the needle is moved or an insertion direction of the needle is changed, and prevents it from being easily removed from a body.

The tasks of the present disclosure are not limited to the tasks mentioned above, and other unmentioned tasks may be clearly understood by those skilled in the art from the description below.

A cannula fixing device for insertion into a body includes a needle having a specific length, and inserted into the body, a connection part connecting a first tube that supplies a solution to the needle, and the needle, and an expansion part that is selectively expanded from an outer peripheral surface of the needle inserted into the body.

The cannula fixing device may further include a second tube connected to the expansion part, and that injects air from an outside.

The expansion part may be disposed to be closely attached to the outer peripheral surface of the needle to surround the outer peripheral surface before the air is injected from the second tube, and to be closely attached to an inner wall of the body as a volume thereof increases when the air is injected from the second tube.

The second tube may be disposed to pass through the connection part or to be coupled thereto through interference fitting.

The expansion part may include a first end being adjacent to a tip end of the needle, and a second end disposed adjacent to an inner wall of the body when the needle is inserted into the body, and the first end may include a temporary contact area temporarily attached to a surface facing the outer peripheral surface of the needle.

The temporary contact area may dissolve when the needle is inserted into the body or separates the needle and the first end by using an expansion pressure of the expansion part.

The expansion part may be coupled to the needle in an area being adjacent to the second end.

The expansion part may be disposed to be spaced apart from the connection part toward a tip end of the needle in a range of 0.3 mm to 1 mm.

The expansion part may include an internal expansion area that is expanded while being inserted into the body on the needle, an external expansion area that is expanded outside the body, and a communication area connecting the internal expansion area and the external expansion area.

The second tube may be connected to the external expansion area.

The expansion part may be expanded to the internal expansion area through the communication area simultaneously when the air is injected into the external expansion area through the second tube.

The communication area may include a communication passage that delivers the air between the external expansion area and the internal expansion area.

A volume increase of the communication passage may be smaller than that of the internal expansion area or the external expansion area.

Specific details of other embodiments are included in the detailed description and drawings.

### BRIEF DESCRIPTION OF THE FIGURES

The summary described above as well as the detailed description of preferred embodiments of the present disclosure that will be made below will be better understood when read in relation to the accompanying drawings. The preferred embodiments are illustrated in the drawings for the purpose of illustrating the present disclosure. However, it should be understood that the present disclosure is not limited to the precise disposition and means illustrated:

FIGS. 1A and 1B are reference views illustrating a state, in which an infusion port is installed in an eyeball according to the present disclosure;

FIG. 2 is a cross-sectional view illustrating a state before an expansion part of a cannula fixing device for an insertion into a body is expanded according to a first embodiment of the present disclosure;

FIG. 3 is a cross-sectional view illustrating a state, in which an expansion part of the cannula fixing device for insertion into a body illustrated in FIG. 2 is expanded;

FIG. 4 is an enlarged cross-sectional view illustrating a cannula fixing device for insertion into a body illustrated in FIG. 3;

FIG. 5 is a reference view illustrating a state before a cannula fixing device for insertion into a body is expanded according to a second embodiment of the present disclosure; and

FIG. 6 is a cross-sectional view illustrating a state, in which a cannula fixing device for insertion into a body illustrated in FIG. 5 is expanded.

### DETAILED DESCRIPTION

Hereinafter, preferred embodiments of the present disclosure will be described in detail with reference to the accompanying drawings. The advantages and features of the present disclosure, and a method for achieving them will become clear with reference to the embodiments that will be described in detail together with the accompanying drawings. However, the present disclosure is not limited by the embodiments disclosed hereinafter but may be implemented in various different forms, and the embodiments are provided simply to make the disclosure of the present disclosure complete and inform an ordinary person in the art of the scope of the present disclosure, and the disclosure is only defined by the scope of the claims. Throughout the specification, the same reference numerals refer to the same elements.

The present disclosure may have various modifications and embodiments, and thus specific embodiments are illustrated and described in the drawings.

However, this is not intended to limit the present disclosure to specific embodiments, but should be understood to include all modifications, equivalents, and substitutes included in the spirit and technical scope of the present disclosure.

Terms including ordinal numbers such as first, second, etc. may be used to describe various elements, but the elements are not limited by the terms.

The terms are used only for the purpose of distinguishing one element from another.

For example, without departing from the scope of the present disclosure, a second element may be referred to as a first element, and similarly, a first element may also be referred to as a second element.

The term “and/or” includes a combination of a plurality of related described items or any one of a plurality of related described items.

When a component is referred to as being “connected” or “electrically connected” to another component, it should be understood that it may be directly connected or electrically connected to that other component, but that there may be other components therebetween.

On the other hand, when a component is referred to as being “directly connected” or “directly electrically connected” to another component, it should be understood that there are no other components therebetween.

The terms used in the application are used only to describe specific embodiments and are not intended to limit the present disclosure.

A singular expression includes a plural expression unless the context clearly indicates otherwise.

When the terms, such as “comprise” and/or “comprising”, is used in the specification, it should be understood that they specify presence of the above-mentioned features, numbers, steps, operations, components, parts, and/or combinations thereof, and do not exclude presence or addition of one or more other numbers, steps, operations, components, parts, and/or combinations thereof.

Hereinafter, embodiments will be described in detail with reference to the accompanying drawings, but the same or corresponding components will be given the same reference number regardless of the reference numerals, and a repeated description thereof will be omitted.

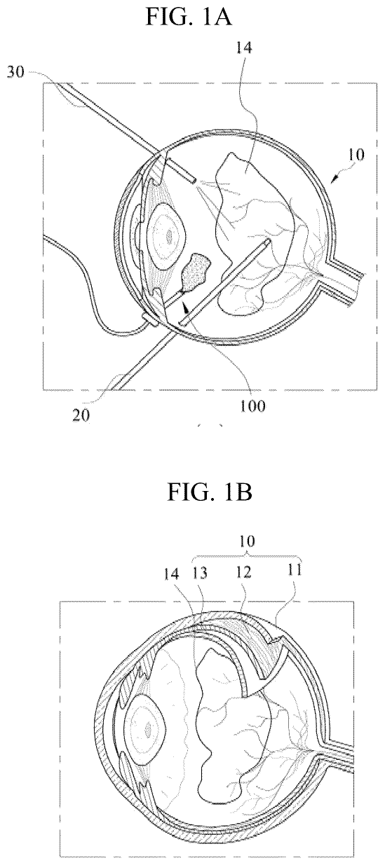

FIGS. 1A and 1B are reference views illustrating a state, in which an infusion port is installed in an eyeball according to the present disclosure.

In FIG. 1A, it is illustrated that various equipment for implementing vitrectomy is located in an interior of an eyeball.

In vitrectomy, an infusion port, on which a cannula fixing device 100 for insertion into a body is mounted, a vitreous body resection device 20 that irradiates light to the interior of an eyeball 10, and a light irradiation part 30 may be used.

A description related to the cannula fixing device 100 for insertion into a body and the infusion port will be described later.

The vitreous body resection device 20 may cut the vitreous body, and vacuum-suction foreign substances together with the cut vitreous body.

Furthermore, the light irradiation part 30 may irradiate light that is required for a surgery to the interior of the eyeball.

The infusion port, the light irradiation part, and the vitreous body resection device may enter the eyeball through a minimal incision on the eyeball, and after the surgery is completed, the incision area may be sutured or finished with no suture.

FIG. 1B briefly illustrates a structure of the eyeball, into which the infusion port is inserted.

First, a cornea, an iris, a lens, and ciliary muscles are disposed in front of the eyeball 10, to which light is introduced, the vitreous body 14 is filled on a rear side thereof, and a retina 13, a choroid 12, and a sclera 11 are disposed outside the vitreous body 14 from an inside to an outside.

Here, the instruments that are required for a surgery are inserted after the sclera 11, the choroid 12, and the retina 13, which are spaced apart from the lens at a specific interval, are sequentially perforated or incised.

First, the sclera 11 is an outermost part of the eyeball 10, and is a tough white fibrous tissue that occupies ⅚ of the back, except for the cornea, muscles that are responsible for movement of the eye is attached thereto, and may allow the tissues that are very dense to maintain the shape of the eye and may protect the eye from an external impact. Often, the white of the eye we see may be a reflection of the sclera through a transparent conjunctiva.

Furthermore, the choroid 12 has a thickness of 0.3 mm to 0.5 mm, and blood vessels are best distributed therein, in the vascular membranes, so that it supplies nutrition to pigment layers or visual cell layers of the retina. It is dark brown because many melanocytes are distributed, and this acts as a darkroom to absorb light from the outside and prevent it from being dispersed toward the sclera. An outer surface of the choroid is loosely coupled to an inner surface of the sclera, and the inner surface is closely attached to a pigment layer of the retina. The choroid is not distributed in a part, in which optic nerves come out behind the eyeball.

Furthermore, the retina 13 is the innermost membrane of the eye formed of several layers and contains several nerve cells and photoreceptor cells so that it senses light, processes and integrates visual information, and transmits it to the brain through the optic nerves.

The human eye is wrapped with a conjunctiva on the outermost side, and is formed of a sclera and a choroid just inside. The sclera changes from the front of the eye to a transparent cornea, allowing light to enter the eye. Retina that is just inside the choroid forms the innermost layer of the eyeball, and has nerve cells of several layers and photoreceptor cells that are cells that sense light waves.

Furthermore, the vitreous body 14 is also called a corpus vitreum, and is a structure that occupies the largest volume, among the contents of the eyeball and exists in the form of a transparent gel while filling a space at the center of the eyeball. Because it plays an important role in maintaining the structure of the eyeball and is transparent, the light that enters through the pupil may safely reach the retina.

Water makes up 98 to 99% of the components of vitreous body and is usually present in the form of an extracellular matrix. The vitreous body has reticular tissue of collagen fibers to form a gel, and the spaces between the collagen fibers are filled with glycosaminoglycans such as hyaluronic acid, so that they serve to maintain the volume of the vitreous body. As we age, these normal structural changes occur, and liquefaction proceeds, in which the gel-like structure gradually changes like water.

The vitreous body 14 maintains a normal shape of the eyeball by filling the space in the eyeball 10, and is optically transparent like the cornea or the lens, allowing the image of light or an object entering the pupil to be formed on the retina.

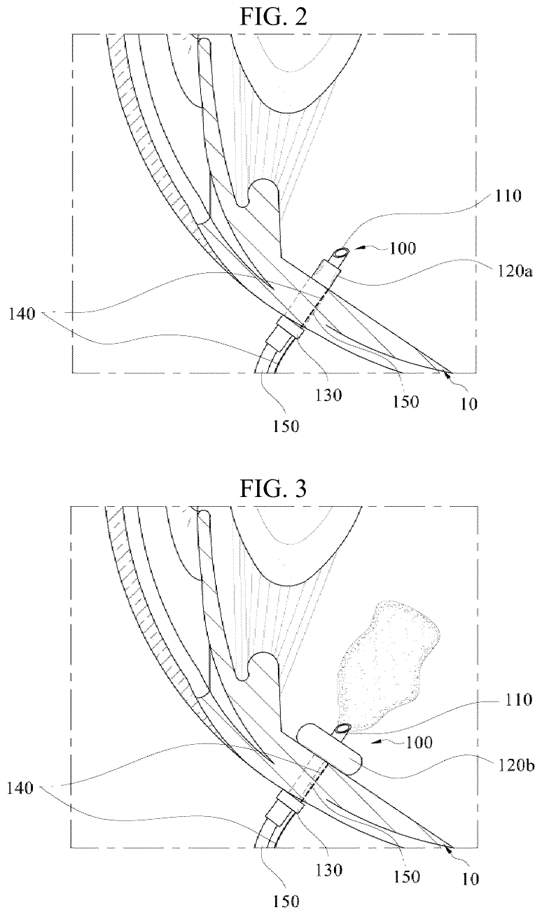

FIG. 2 is a cross-sectional view illustrating a state before an expansion part of a cannula fixing device for an insertion into a body is expanded according to a first embodiment of the present disclosure, and FIG. 3 is a cross-sectional view illustrating a state, in which an expansion part of the cannula fixing device for insertion into a body illustrated in FIG. 2 is expanded.

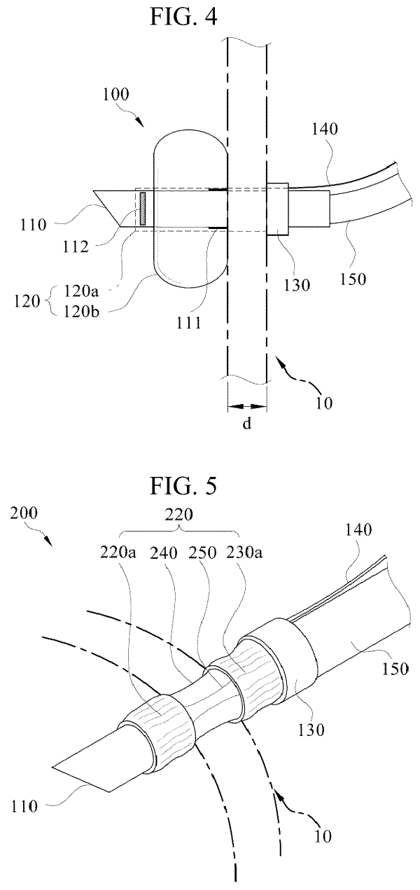

Referring to FIGS. 2 and 3, a cannula fixing device 100 for insertion into a body according to a first embodiment of the present disclosure may include a needle 110, an expansion part 120 (see FIG. 4), and a connection part 130. The cannula fixing device 100 for insertion into a body may be the above-described infusion port, or may be a partial configuration of the infusion port.

The needle 110 may be a needle that is conventionally used for an infusion port, or a pipe-shaped tube having a non-pointed tip end may be used. In this case, a depth of the needle that may be inserted into the eyeball 10 may be formed to be about 4 mm.

The needle 110 may be inserted to form a right angle with respect to an outer peripheral surface of the eyeball 10. Of course, in some cases, it may be inserted obliquely into the eyeball 10.

Furthermore, the connection part 130 provides a function of connecting the needle 110 on one side and a first tube 150 on an opposite side, and may be integrally formed with the needle 110. It is preferable that the connection part 130 is disposed outside the eyeball 10 even after the insertion of the needle 110 is completed.

A first tube 150 for supplying a solution may be coupled to an opposite side of the connection part 130. The first tube 150 may deliver physiological saline supplied from the outside to the needle 110 through the connection part 130.

Although not illustrated in the drawing, the connection part 130 may be formed in a connection pipe shape such that the needle 110 is coupled to one side thereof and the first tube 150 is coupled to an opposite side thereof. Furthermore, a contact preventing layer (not illustrated) formed of a silicone or rubber material may be provided on the surface, on which the connection part 130 and the eyeball 10 contact each other to prevent damage caused by the contact with the eyeball 10. The contact preventing layer may be attached, coated, or deposited on one side of the connection part 130 that faces the eyeball 10.

Furthermore, the expansion part 120 may be formed in a donut-shaped tube structure formed of a thin film, of which an internal volume thereof may be increased. The expansion part 120 may be coupled to the outer peripheral surface of the needle 110, and may be introduced into the eyeball together when the needle 110 is inserted into the eyeball 10.

A second tube 140 may be connected to the expansion part 120.

The second tube 140 may deliver air that will be injected into the expansion part 120 from the outside. Because the second tube 140 is disposed to be closely attached to the needle 110 and has a micro-tubular shape, a perforation diameter may not be considered during the process of perforating the eyeball 10.

The expansion part 120 inserted into the eyeball 10 may receive air from the second tube 140 at the time when the insertion of the needle 110 is completed and may expand as illustrated in FIG. 3. When the volume increases while air is injected into the expansion part, it may be disposed to be closely attached to the inner wall of the eyeball 10 (e.g., the inner wall of the membrane) while being close attached to the outer peripheral surface of the needle.

Accordingly, the expansion part 120 may provide an effect of preventing the needle 110 from being separated from the eyeball 10 and preventing an insertion direction of the needle 110 from being distorted.

Furthermore, the second tube 140 may be disposed on the connection part 130 to pass therethrough adjacent to the first tube 150. Alternatively, the second tube 140 may be assembled in a structure that is coupled to a side surface of the connection part 130 through interference-fitting.

FIG. 4 is an enlarged cross-sectional view illustrating a cannula fixing device for insertion into a body illustrated in FIG. 3.

Referring to FIG. 4, the expansion part may include a first end that is adjacent to the tip end of the needle 110 in a state before the expansion part is expanded (hereinafter, referred to as a contracted expansion part 120a) and a second end that is disposed adjacent to the inner wall of the eyeball 10 when the needle is inserted into the eyeball 10.

In this case, the contracted expansion part 120a may include a temporary contact area 112 that is temporarily adhered or attached to the outer peripheral surface of the needle 110 adjacent to the first end. For example, the temporary contact area 112 may be an area, in which the contracted expansion part 120a is adhered to the outer peripheral surface of the needle 110 by a medical adhesive, and then dissolves after being inserted into the eyeball to release the adhesion force. This may be expected to facilitate the insertion of the contracted expansion part 120a when the needle 110 is inserted into the eyeball 10.

Furthermore, the temporary contact area 112 may be applied as a structure, in which the contracted expansion part 120a is temporarily attached by heat while being coupled to the needle outer peripheral surface, or is fixed adjacent to the first end as a ring or hook-shaped catching part is provided on the contracted expansion part 120a, and thereafter, it is inserted into the interior of the eyeball 10 and then a part adhered by the heat is separated by an expansion pressure of the expansion part or the temporary contact area 112 is separated while the structure of the catching part is damaged.

However, regardless of the contracted or expanded state, it is preferable that the expansion part 120 continuously maintains an attachment area 111 that is adjacent to the second end, coupled or attached to the needle 110.

Furthermore, as illustrated in FIG. 4, when the insertion direction of the needle is distorted or curved in the interior of the eyeball 10, the needle 110 and the expanded expansion part 120b may be disposed at the tip end of the needle 110 to prevent a sharp area from contacting the inner wall of the eyeball 10. To this end, considering that an insertion length of the needle 110 is about 4 mm and a diameter of the needle 110 is in a range of 0.5 mm to 0.6 mm, it may be designed to have a height of about 2 mm and a diameter of about 4 mm when the expansion part 120b is expanded.

Furthermore, a gap “d” may be formed such that the expansion part 120 is spaced apart from the connection part toward the tip end of the needle in the range of 0.3 mm to 1 mm. For example, the gap “d” between the second end of the expansion part 120 and the connection part 130 may correspond to a thickness of the eyeball 10. Of course, the gap “d” between the second end of the expansion part 120 and the connection part 130 may have a size that is at least equal to or greater than the thickness of the eyeball 10.

Furthermore, although not illustrated in the drawing, LED silicon having a light emitting effect may be applied to the expansion part 120. When the expansion part 120 is applied by LED silicon, the above-described light irradiation part (see FIG. 1) is not necessary, and thus an effect of reducing the perforation area may be expected. Furthermore, when LED silicon is applied together with the light irradiation part, illumination in the three-dimensional eyeball is possible, and thus shadow by the surgical instrument in the eyeball may be decreased, and thus an effect of securing a more efficient surgical field of view may be expected.

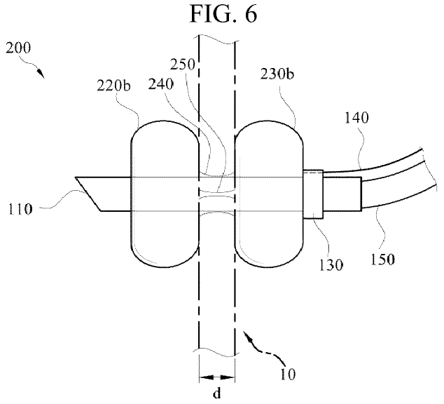

FIG. 5 is a reference view illustrating a state before a cannula fixing device for insertion into a body is expanded according to a second embodiment of the present disclosure, and FIG. 6 is a cross-sectional view illustrating a state, in which a cannula fixing device for insertion into a body illustrated in FIG. 5 is expanded.

A cannula fixing device for insertion into a body 200 according to the second embodiment of the present disclosure has a difference in the structure of an expansion part 220. Hereinafter, the same reference numerals as the reference numerals described above may denote the same elements.

The expansion part 220 according to the second embodiment may include internal expansion areas 220a and 220b, external expansion areas 230a and 230b, and a communication area 240. Here, the internal expansion area and the external expansion area may be divided into a contracted internal expansion area 220a, a contracted external expansion area 230a, an expanded internal expansion area 220b, and an expanded external expansion area 230b.

The internal expansion areas 220a and 220b may have a structure that is similar to that of the expansion part 120 according to the first embodiment described above. The internal expansion areas 220a and 220b may be inserted together while the needle 110 is inserted into the eyeball. Of course, although not illustrated in the drawings, an attachment structure to the needle 110 of the contracted expansion part 120a, a length relationship between the needle 110 and the expanded expansion part 120b in the first embodiment may be equally applied to the internal expansion areas 220a and 220b.

Furthermore, the external expansion areas 230a and 230b may be located in an area between the outer wall of the eyeball 10 and the connection part 130. Furthermore, the second tube may be connected to the external expansion areas 230a and 230b.

Furthermore, the communication area 240 may be connected to communicate the internal expansion areas 220a and 220b and the external expansion areas 230a and 230b. Accordingly, when air is injected through the second tube 140, the air may be injected throughout the expansion part 220 from the external expansion areas 230a and 230b to the internal expansion areas 220a and 220b via the communication area 240.

In this case, the communication area 240 may include a communication passage 250 for delivering air between the internal expansion areas 220a and 220b and the external expansion areas 230a and 230b. Although one communication passage 250 is described as an example in the drawing, a plurality of communication passages 250 may be formed to be disposed at equal intervals or at equal angles on the communication area 240.

Of course, the communication area 240 may be made of a material with relatively little volume increase or no volume increase compared to the internal expansion areas 220a and 220b and the external expansion areas 230a and 230b.

In this way, when the expansion part 220 is provided with inner expansion areas 220a and 220b and outer expansion areas 230a and 230b around the sidewall of the eyeball 10, the needle 110 may be prevented from being inclined or curved on the sidewall of the eyeball 10, friction damage in an area closely attached to the eyeball 10 may be minimized inside/outside the eyeball 10, and it is possible to more reliably maintain the internal pressure in the area perforated in the eyeball 10 by the needle 110. Furthermore, an effect of simplifying the structure of connecting the second tube 140, and stably connecting it may be accompanied.

Accordingly, according to the cannula fixing device for insertion into a body according to an embodiment of the present disclosure, it is possible to stably support the needle inserted into the eyeball, damage to the inner wall of the eyeball may be prevented even though the needle is inclined or curved inside the eyeball, it is possible to prevent the pressure from escaping from the area perforated in the eyeball while inserting the needle, medical accidents that occur when the needle is separated from the eyeball may be prevented in advance.

Although the present disclosure has been described with reference to the embodiments, it will be appreciated by an ordinary skilled in the art, to which the present disclosure pertains, that the present disclosure may be modified and changed within the scope of the appended claims without departing from the spirits and technical field of the present disclosure. Accordingly, such modifications should also be considered to fall within the scope of the present disclosure, and the scope of the present disclosure should be determined by the claims set forth below.

What is claimed is:

1. A cannula fixing device for insertion into a body, the cannula fixing device comprising:
a needle having a specific length, and inserted into the body;
a connection part connecting a first tube configured to supply a solution to the needle, and the needle; and
an expansion part configured to be selectively expanded from an outer peripheral surface of the needle inserted into the body.

2. The cannula fixing device of claim 1, further comprising:
a second tube connected to the expansion part, and configured to inject air from an outside.

3. The cannula fixing device of claim 2, wherein the expansion part is disposed to be closely attached to the outer peripheral surface of the needle to surround the outer peripheral surface before the air is injected from the second tube, and to be closely attached to an inner wall of the body as a volume thereof increases when the air is injected from the second tube.

4. The cannula fixing device of claim 2, wherein the second tube is disposed to pass through the connection part or to be coupled thereto through interference fitting.

5. The cannula fixing device of claim 1, wherein the expansion part includes:
a first end being adjacent to a tip end of the needle; and
a second end disposed adjacent to an inner wall of the body when the needle is inserted into the body, and
wherein the first end includes a temporary contact area temporarily attached to a surface facing the outer peripheral surface of the needle.

6. The cannula fixing device of claim 4, wherein the temporary contact area dissolves when the needle is inserted into the body or separates the needle and the first end by using an expansion pressure of the expansion part.

7. The cannula fixing device of claim 4, wherein the expansion part is coupled to the needle in an area being adjacent to the second end.

8. The cannula fixing device of claim 1, wherein the expansion part is disposed to be spaced apart from the connection part toward a tip end of the needle in a range of 0.3 mm to 1 mm.

9. The cannula fixing device of claim 2, wherein the expansion part includes:
an internal expansion area configured to be expanded while being inserted into the body on the needle;
an external expansion area configured to be expanded outside the body; and
a communication area connecting the internal expansion area and the external expansion area.

10. The cannula fixing device of claim 9, wherein the second tube is connected to the external expansion area.

11. The cannula fixing device of claim 10, wherein the expansion part is expanded to the internal expansion area through the communication area simultaneously when the air is injected into the external expansion area through the second tube.

12. The cannula fixing device of claim 10, wherein the communication area includes:
a communication passage configured to deliver the air between the external expansion area and the internal expansion area.

13. The cannula fixing device of claim 12, wherein a volume increase of the communication passage is smaller than that of the internal expansion area or the external expansion area.
